(19) United States
(12) Patent Application Publication
(10) Pub. No.: US 2024/0165055 A1
(43) Pub. Date: May 23, 2024

(54) TREATMENT

(71) Applicant: Verona Pharma PLC, Cardiff (GB)

(72) Inventors: Tara Renae RHEAULT, Cary, NC (US); Kathleen RICKARD, Durham, NC (US); Thomas BENGTSSON, Lund (SE)

(21) Appl. No.: 18/424,468

(22) Filed: Jan. 26, 2024

Related U.S. Application Data
(63) Continuation of application No. PCT/GB2023/052083, filed on Aug. 7, 2023.
(60) Provisional application No. 63/502,977, filed on May 18, 2023.
(60) Provisional application No. 63/370,699, filed on Aug. 8, 2022.
(60) Provisional application No. 63/370,696, filed on Aug. 8, 2022.
(60) Provisional application No. 63/370,694, filed on Aug. 8, 2022.

(51) Int. Cl. A61K 31/137; A61K 9/00; A61K 31/519; A61K 31/573; A61K 47/26; A61P 11/08

(52) U.S. Cl. CPC A61K 31/137; A61K 9/0078; A61K 31/519; A61K 31/573; A61K 47/26; A61P 11/08

(57) ABSTRACT

The present invention relates to a compound for use in a method of increasing trough lung function in a patient suffering from chronic obstructive pulmonary disease (COPD), wherein the compound is ensifentrine or a pharmaceutically acceptable salt thereof. The invention also relates to a compound for use in treating chronic obstructive pulmonary disease (COPD) in a patient, wherein: the compound is ensifentrine or a pharmaceutically acceptable salt thereof and the patient is susceptible to disturbed sleep.

### CROSS REFERENCE

This application is a bypass continuation of International Application No. PCT/GB2023/052083, filed on Aug. 7, 2023, which claims priority to U.S. Provisional Application No. 63/502,977, filed on May 18, 2023, U.S. Provisional Application No. 63/370,699, filed on Aug. 8, 2022, U.S. Provisional Application No. 63/370,696, filed on Aug. 8, 2022, and U.S. Provisional Application No. 63/370,694, filed on Aug. 8, 2022, each of which is incorporated herein by reference in its entirety.

### FIELD OF THE INVENTION

The present invention relates to increasing trough lung function in a patient suffering from chronic obstructive pulmonary disease (COPD). The present invention also relates to the treatment of COPD in patients susceptible to disturbed sleep.

### BACKGROUND OF THE INVENTION

Ensifentrine (N-(2-{(2E)-9,10-dimethoxy-4-oxo-2-[(2, 4,6-trimethylphenyl)imino]-6,7-dihydro-2H-pyrimido[6,1-α]isoquinolin-3(4H)-yl}ethypurea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

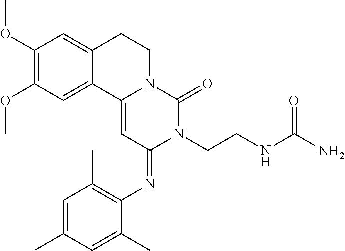 [SMILES: COc1cc2c(cc1OC)-c1c/c(=N\c3c(C)cc(C)cc3C)n(CCNC(N)=O)c(=O)n1CC2]

COPD is a progressive, long-term condition affecting a large number of people worldwide. Ongoing symptoms include breathlessness and cough. Treatment of COPD typically comprises maintenance treatment in which a patient is administered a drug on a regular basis (for instance once or twice daily) to improve lung function and ameliorate the symptoms of COPD.

The lung function of a COPD patient typically follows a cycle during maintenance therapy. Lung function in COPD can be measured by determining the forced expiratory volume in one second (FEV₁) of a patient. In the period following administration of the drug, improved lung function is achieved, reaching a peak improvement a certain period after the drug was administered. The lung function of the patient then decreases towards the trough lung function: i.e. the lowest point in the lung function cycle during maintenance therapy. The trough lung function generally occurs shortly before the next administration of the maintenance therapy drug. For instance, for a twice daily (morning and evening) maintenance therapy, trough lung function generally occurs immediately before the morning dose of the drug (morning trough lung function) and evening dose of the drug (evening trough lung function).

The trough lung function achieved during a maintenance therapy can have significant effects on the symptoms and/or quality of life of a COPD patient. The trough lung function effectively represents the worst lung function at a stage during the maintenance treatment. For a twice daily dosing, the morning trough lung function will often coincide with a period of time for which the patient is asleep. This means that the patient can have poor lung function for part of the night, disrupting his or her sleep (for instance due to limited oxygen being absorbed) and causing tiredness, thereby reducing quality of life. This is particularly the case in COPD patients who already have disturbed sleep, for instance due to sleep disorders and co-morbidities that disrupt sleep.

If trough lung function can be improved, then symptoms of COPD can be more consistently ameliorated and quality of life. Improvement in morning trough lung function can be particularly beneficial in those patients already susceptible to sleep disruption.

There are a number of drugs disclosed for use in treating COPD. However, they are not all equally effective specifically in improving trough lung function, and in particular morning trough lung function. It would be clinically advantageous to administer specific drugs which are particularly effective in improving trough lung function to patients for whom that is a specific therapeutic desiderata, to optimise the therapeutic effect of the pharmacological intervention.

### SUMMARY OF THE INVENTION

It is a finding of the present invention that ensifentrine is particularly effective in increasing trough lung function in patients with COPD when used as a maintenance therapy. In particular, ensifentrine can improve morning trough lung function in COPD patients, which is particularly beneficial for COPD patients who are susceptible to sleep disruption, for instance due to the presence of a co-morbidity which affects sleep.

The invention accordingly provides a compound for use in a method of increasing trough lung function in a patient suffering from chronic obstructive pulmonary disease (COPD), wherein the compound is ensifentrine or a pharmaceutically acceptable salt thereof

Also provided by the invention is a compound for use in treating chronic obstructive pulmonary disease (COPD) in a patient, wherein: the compound is ensifentrine or a pharmaceutically acceptable salt thereof; and the patient is susceptible to disturbed sleep.

The invention also provides a method of increasing trough lung function in a patient suffering from COPD, the method comprising administering a therapeutically effective amount of a compound to the patient, which compound is ensifentrine or a pharmaceutically acceptable salt thereof.

The invention further provides a method of treating COPD in a patient, which method comprises administering a therapeutically effective amount of a compound which is ensifentrine or a pharmaceutically acceptable salt thereof to the patient, wherein the patient is susceptible to disturbed sleep.

Further provided by the invention is use of a compound in the manufacture of a medicament for use in a method of increasing trough lung function in a patient suffering from COPD, wherein the compound is ensifentrine or a pharmaceutically acceptable salt thereof

Also provided by the invention is use of a compound which is ensifentrine or a pharmaceutically acceptable salt thereof in the manufacture of a medicament for treating COPD, wherein the patient is susceptible to disturbed sleep.

### BRIEF DESCRIPTION OF THE FIGURES

FIG. 1 shows the FEV₁ profile over 12 hours at Week 12.

### DETAILED DESCRIPTION OF THE INVENTION

The patient is typically receiving the compound as a maintenance therapy. The compound may be administered to the patient once, twice or three times daily. The compound is preferably administered as a twice-daily maintenance therapy.

An increase in trough lung function in a patient suffering from COPD is typically determined by measuring in increase in trough FEV₁, i.e. the FEVi of the patient shortly before administration of the compound as part of the maintenance therapy. The improvement in trough lung function may for instance arise from a change in maintenance therapy drug.

The method may comprises increasing morning trough lung function (i.e. trough lung function following sleep). Morning trough lung function can be measured by determining the FEVi of the patient shortly before the morning administration of the compound as part of the maintenance therapy. For instance, FEV₁ may be measured less than an hour before the morning administration of the compound. Morning trough FEV₁ may be the FEV₁ as measured between 11.5 and 12 hours following the prior evening dose.

Typically, as used herein, FEV₁ and FVC (forced vital capacity) are determined as set out in the article Standardisation of spirometry, Eur J 2005; 26; 319-338.

Improvement in trough lung function can improve sleep in a COPD patient. This is particularly important in a patient who is susceptible to disturbed sleep. Patients susceptible to disturbed sleep typically suffer from a condition that directly affects their ability to sleep (for instance insomnia or sleep apnea) or from a condition that indirectly makes it harder to sleep (for instance skin conditions such as psoriasis that irritate the skin and make it harder for a patient to fall asleep or to remain asleep).

A “patient susceptible to disturbed sleep” is typically a patient suffering from one or more disease or condition selected from obesity, insomnia, sleep apnea, narcolepsy, restless leg syndrome, REM sleep behavious disorder, circadian rhythm sleep disorders parasomnias, depression, anxiety, psoriasis, dermatitis, eczema, or urticaria. For instance, the compound may be for use in treating COPD in a patient suffering from COPD and sleep apnea. The compound may be for use in treating COPD in a patient suffering from COPD and a skin condition such as psoriasis, dermatitis, eczema, or urticaria.

Improved trough lung function can significantly assist a COPD patient in exercising. Thus, in a preferred embodiment, the patent is suffering from obesity.

The patient may be male. The patient may be female. The patient may have an age of greater than or equal to 65 years. The patient may have an age of less than 65 years. The patient may be taking a background medication selected from one or more of a long-acting muscarinic antagonist (LAMA), a long-acting beta-agonist (LABA) and an inhaled corticosteroid (ICS).

The compound is ensifentrine or a pharmaceutically acceptable salt thereof. Pharmaceutically acceptable salts are well known to the skilled person. Typically, the compound is ensifentrine (i.e. ensifentrine free base).

The method typically comprises administering the compound to the patient by inhalation. A pharmaceutical composition comprising the compound and one or more pharmaceutically acceptable excipients or diluents is typically administered to the patient by inhalation, for instance by nebuliser, pressurised metered dose inhaler (pMDI) or dry powder inhaler (DPI).

Preferably, the method comprises administering the compound to the patient by inhalation from a nebuliser. Nebulisers aerosolise a liquid pharmaceutical composition into an aerosol that is inhaled into a patient's respiratory tract. Examples of nebulisers include a soft mist nebuliser, a vibrating mesh nebuliser, a jet nebuliser and an ultrasonic wave nebuliser. Suitable nebuliser devices include the Philips I-neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The compound may be inhaled via the nebuliser for from 1 to 15 minutes.

Typically, the method comprises administering the compound to the patient once, twice or three times per day, for instance twice or three times per day. The compound may be administered to the patient by inhalation once, twice or three times a day. Preferably the method comprises administering the compound to the patient by inhalation twice a day. The method may comprise administering a first dose of the compound in the morning (for instance within 3 hours following waking) and a second dose of the compound in the evening (for instance within 3 hours before bed). Typically, the morning and evening doses are administered from 10 to 14 hours apart, for instance about 12 hours apart.

The compound may be used in any suitable therapeutically effective amount. Typically, the daily dose of the compound is from 0.1 to 20 mg. Typically, the method comprises administering a total daily dose of the compound of from 0.5 to 10 mg. Preferably, the total daily dose of the compound (e.g. ensifentrine free base) is from 5 to 7 mg, for instance about 6 mg per day. As used herein, the term “about” may represent a variation of ±10% of the stated value. The total daily dose of the compound may be 6.0 mg.

Typically, the compound is administered twice a day in two separate doses which are the same or similar. For instance, the method may comprise administering the compound to the patient twice a day in a first dose of from 1 to 5 mg and a second dose of from 1 to 5 mg. Typically, the method may comprise administering the compound to the patient twice a day in a first dose of from 2 to 4 mg and a second dose of from 2 to 4 mg.

Preferably, the method comprises administering two doses of about 3 mg ensifentrine free base to the patient per day by inhalation. The method preferably comprises administering a dose of about 3 mg of the compound to the patient twice a day (3 mg BID) by inhalation. More preferably, the method comprises administering by nebuliser a dose of about 3 mg the compound to the patient twice a day. Each dose may be 3.0 mg free base ensifentrine administered by nebulizer.

The compound is typically used as a maintenance therapy. Typically, the method comprises administering the compound to the patient at least once per day for at least 8 weeks. The compound may be administered to the patient at least once per day for at least 16 weeks, preferably for at least 24 weeks. The compound may be administered daily to the patient for at least 1 year. The method may comprise administering the compound to the patient at least once every 24 hours, preferably at least twice every 24 hours, for at least 8 weeks, preferably for at least 16 weeks, more preferably for at least 24 weeks.

The compound is preferably administered as a suspension formulation, i.e. a suspension of particles comprising the compound in a diluent. The compound may alternatively be delivered as a dry powder, for instance a dry powder comprising particles comprising the compound and particles of a carrier such as lactose.

The method typically comprises administering an inhalable pharmaceutical composition comprising a suspension of particles of the compound in a diluent. The particles comprising the compound typically have a particle size distribution with a Dv50 of from 0.5 vim to 5.0 μm. The particles preferably have a Dv50 of from 1.0 μm to 2.0 μm.

Particle sizes are described herein by reference to the Dv50 value, which is the median particle size for a volume distribution. Thus, half the volume of the particles have diameters of less than the Dv50 value and half the volume of the particles have diameters of greater than the Dv50 value. This is a well-known manner in which to describe particle size distributions.

The technique used to measure the Dv50 values as stated herein is typically laser diffraction. The particle size distribution of the particles comprising the compound may be as measured by laser diffraction using a wet powder dispersion system. For instance, the particle size distribution can be measured by laser diffraction using a Malvern Spraytec in conjunction with a wet dispersion cell. Typically, the instrument parameters for the Malvem Spraytec are as follows:
particle—standard opaque particle;
refractive index Particle—1.50;
refractive index (imaginary)—0.50;
density of particle—1.00;
refractive index of dispersant—1.33;
controller unit—1000RPM;
measurement type—timed;
initial sampling time—30s;
obscuration—20% -30%;
dispersant—1% Polysorbate 20 in deionised water.

The particles comprising the compound typically comprise ensifentrine (i.e. ensifentrine free base). The particles may comprise at least 90 wt % ensifentrine free base relative to the total weight of the particles. The particles may comprise at least 99 wt % ensifentrine. The particles may consist of ensifentrine.

The concentration of particles comprising the compound in the inhalable pharmaceutical composition is typically from 0.1 to 5.0 mg/mL, preferably from 0.1 to 2.5 mg/mL, more preferably from 1.0 to 2.0 mg/mL.

The inhalable pharmaceutical composition typically further comprises one or more tonicity adjusters, one or more buffers and one or more surfactants. The tonicity adjuster is typically sodium chloride.

Examples of buffers include a citrate buffer, a phosphate buffer, an acetate buffer, and a bicarbonate buffer. Preferably, the buffer is a phosphate buffer, for instance sodium dihydrogen phosphate dihydrate and/or disodium phosphate dihydrate.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

Preferably, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). Preferably, the sterile liquid vehicle comprises polysorbate 20 and/or sorbitan monolaurate (Span 20).

For instance, the method may comprise administering to the patient an inhalable liquid pharmaceutical composition comprising:
water;
particles consisting of ensifentrine free base at a concentration of from 0.1 to 20 mg/mL;
one or more tonicity adjusters at a total concentration of from 1.0 to 15 mg/mL;
one or more buffers at a total concentration of from 0.1 to 4 mg/mL; and
one or more surfactants at a total concentration of from 0.05 to 3 mg/mL.

The inhalable liquid pharmaceutical composition may comprise:
water;
particles consisting of ensifentrine free base at a concentration of from 0.5 to 6 mg/mL;
sodium chloride at a concentration of from 5 to 12 mg/mL;
sodium dihydrogen phosphate dihydrate at a concentration of from 0.3 to 2 mg/mL;
disodium phosphate dihydrate at a concentration of from 0.3 to 2 mg/mL;
polysorbate 20 at a concentration of from 0.1 to 1.5 mg/mL; and
sorbitan monolaurate at a concentration of from 0.01 to 0.5 mg/mL.

The compound may be used in combination with a second active agent. The compound may be administered separately or simultaneously with the second active agent. The patient may already be taking a second active agent as a background therapy for COPD. Alternatively, treatment with the second active agent may start at around the same time as treatment with the compound. The compound and the second active agent may be administered in a fixed combination.

The second active agent is typically a muscarinic receptor antagonist, a beta-adrenergic receptor agonist or an inhaled corticosteroid. The compound may accordingly be used in combination with muscarinic receptor antagonist, a beta-adrenergic receptor agonist or an inhaled corticosteroid. The second active agent may be a long-acting muscarinic receptor antagonist (LAMA) or a long-acting beta-adrenergic receptor agonist (LABA).

Examples of LAMAs include aclidinium, darotropium, tiotropium, glycopyrrolate and umeclidinium. Examples of LABAs include salmeterol, formoterol, indacaterol, vilanterol, olodaterol, abediterol and carmoterol. Examples of inhaled corticosteroids include beclomethosone, budesonide, fluticasone propionate, ciclesonide, mometasone and fluticasone furoate.

The patient may be using a beta-agonist (for instance salbutamol) as a rescue medication.

The invention is described in more detail by the following Example.

### EXAMPLES

Study design

A clinical study was conducted to determine the efficacy of ensifentrine in treating COPD compared with placebo. Ensifentrine was administered by nebuliser at a dose of 3 mg twice daily (BID) for 24 weeks. The study was a multi-centre, randomized, double-blind, parallel-group, placebo-controlled trial with around 800 patients and 5:3 randomization.

The study population included patients aged 40-80 years with moderate to severe COPD (FEV₁30%-70% p.n., FEVi/forced vital capacity [FVC] ratio <0.7, with mMRC ≥2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC)0-12h post-dose at week 12. Secondary endpoints of the study included: peak FEV₁ over 4 hours post-dose at Week 12; morning trough FEV₁ at Week 12; and other endpoints including moderate/severe COPD exacerbations frequency over 24 Weeks.

Methods

COPD severity is derived as follows: mild: 80%<=FEV₁, moderate: 50%<=FEV₁<80% predicted, severe: 30%<=FEV₁<50% predicted and very severe: FEV1<0% predicted, post bronchodilator dose at Screening.

Baseline FEV₁ is the mean of the two measurements taken before study medication on the day of first dosing, i.e. ≤40 minutes and just prior to dosing, pre-dose on day 1.

Average FEV₁ AUCO-12h is defined as area under the curve over 12 hours of the FEV₁, divided by 12 hours.

Morning trough FEV₁ at Week 12 is defined as the FEVi assessed at 11.5 to 12 hours following the prior evening dose.

Formulation

The investigational product and placebo were provided in 2.5 mL unit dose format in an ampule and administered via a nebuliser. The formulation of the investigational product (ensifentrine suspension formulation) and placebo are shown in Table 1 below.

TABLE 1

| | Concentration |
|---|---|
| Constituent | (mg/mL) |
| Ensifentrine particles (RPL554) | 1.2 (for |
| | the active) |
| | or 0 (for |
| | Placebo) |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

Results

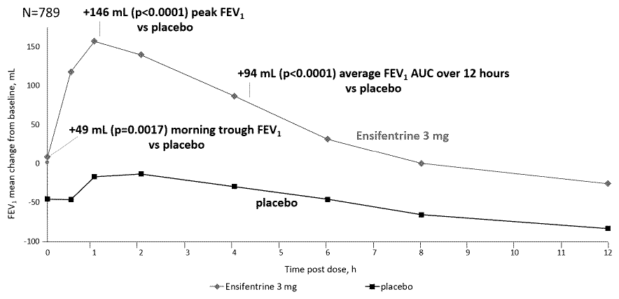

The primary endpoint of average FEV₁ (AUC)0-12h at Week 12 was met. All subgroups showed improvement in lung function with ensifentrine that was statistically significant. The results are shown in Table 2 and FIG. 1.

TABLE 2

| Average FEV₁ (AUC)0-12 h | Ensifentrine | |
|---|---|---|
| post-dose at Week 12: | 3 mg | Placebo |
| Change from Baseline (CFB) | (n = 498) | (n = 291) |
| Observed N | 424 | 231 |
| Mean Baseline FEV₁, L | 1.3323 | 1.2316 |
| CFB Least Squares Mean, | 48.2 (30.0, 66.5) | 45.7 (−69.7, −21.6) |
| mL (95% CI) | | |
| Placebo-corrected CFB LS | 93.9 (64.5, 123.3) | — |
| Mean Diff., mL (95% CI) | | |
| p-value | <0.0001 | — |

The effects of ensifentrine on Morning Trough FEV₁ at Week 12 is shown in Table 3.

TABLE 3

| | Ensifentrine | |
|---|---|---|
| Morning | 3 mg | Placebo |
| Trough FEV₁ Week 12 | (n = 498) | (n = 291) |
| Observed N | 434 | 242 |
| Mean Baseline FEV₁, L | 1.2934 | 1.2386 |
| CFB Least Squares Mean, | 6.3 (−12.3, 24.9) | −42.6 (−67.3, −17.9) |
| mL (95% CI) | | |
| Placebo-corrected CFB LS | 48.9 (18.3, 79.5) | — |
| Mean Diff., mL (95% CI) | | |
| p-value | 0.0017 | — |

Morning trough FEV₁ showed a statistically significant effect at week 12, confirming a twice daily dosing interval.

Conclusion

It has been found that ensifentrine provides a statistically significant improvement in lung function in all subgroups of COPD patients in the study. In addition, ensifentrine has been found to be particularly effective in improving trough lung function, in particular by increasing morning trough FEV₁.

What is claimed is:

1. A method of increasing trough lung function in a human subject having chronic obstructive pulmonary disease (COPD), the method comprising administering to the human subject via inhalation two or more doses of a composition comprising a therapeutically effective amount of ensifentrine or a pharmaceutically acceptable salt thereof, wherein the trough lung function is increased by at least about 40 mL as determined by FEV₁ of the human subject about 11.5 hours to about 12 hours following a prior administration of the composition compared to FEV₁ of the human subject prior to a first administration of the composition to the human subject.

2. The method of claim 1, wherein the composition is administered to the human subject as a maintenance therapy.

3. The method of claim 1, wherein the trough lung function is measured about 12 hours following a prior administration of the composition to the human subject.

4. The method of claim 1, wherein the trough lung function is measured within one hour prior to an administration of the composition to the human subject.

5. The method of claim 1, wherein the COPD is moderate COPD.

6. The method of claim 1, when the COPD is severe COPD.

7. The method of claim 1, wherein the increasing trough lung function reduces sleep disruption in the human subject.

8. The method of claim 1, wherein the trough lung function is increased by at least about 49 mL.

9. The method of claim 1, wherein the administering to the human subject is twice per day in a first dose and a second dose.

10. The method of claim 9, wherein the first dose and the second dose are administered to the human subject in equal amounts.

11. The method of claim 9, wherein the first dose is administered to the human subject in the morning and the second dose is administered to the human subject in the evening.

12. The method of claim 9, wherein the first dose is administered within three hours after the human subject waking, and the second dose is administered within three hours before the human subject sleeps.

13. The method of claim 9, wherein the first dose and the second dose are administered to the human subject from 10 to 14 hours apart.

14. The method of claim 1, wherein the therapeutically effective amount of the ensifentrine or the pharmaceutically acceptable salt thereof is 2 mg to 4 mg.

15. The method of claim 14, wherein the therapeutically effective amount of the ensifentrine or the pharmaceutically acceptable salt thereof is 3 mg.

16. The method of claim 1, wherein the composition is a suspension.

17. The method of claim 16, wherein the suspension comprises particles, wherein the particles comprise at least 90 weight percent ensifentrine relative to total weight of the particles.

18. The method of claim 16, wherein the suspension comprises particles, wherein the particles comprise at least 99 weight percent ensifentrine relative to total weight of the particles.

19. The method of claim 16, wherein the suspension comprises 3 mg ensifentrine free base.

20. The method of claim 16, wherein the suspension comprises particles, wherein the particles have a particle size distribution with a Dv50 of from 0.5μm to 5 gm.

21. The method of claim 16, wherein the suspension comprises particles, wherein the particles have a particle size distribution with a Dv50 of from 1 gm to 2 gm.

22. The method of claim 16, wherein the suspension comprises:
a. ensifentrine particles at a concentration of from 1 to 1.4 mg/mL;
b. polysorbate 20 (Tween 20) at a concentration of from 0.3 to 0.7 mg/mL;
c. sorbitan monolaurate (Span 20) at a concentration of from 0.0 to 0.1 mg/mL;
d. sodium dihydrogen phosphate dihydrate at a concentration of from 0.5 to 1 mg/mL;
e. disodium hydrogen phosphate dihydrate at a concentration of from 0.5 to 1 mg/mL;
f. sodium chloride at a concentration of from 5 to 10 mg/mL; and
g. water.

23. The method of claim 16, wherein the suspension comprises:
a. 1.2 mg/ml ensifentrine;
b. 0.50m g/ml polysorbate 20;
c. 0.05 mg/ml sorbitan monolaurate;
d. 0.744 mg/ml sodium dihydrogen phosphate dihydrate;
e. 0.853 mg/ml disodium hydrogen phosphate dihydrate;
f. 8.60 mg/ml sodium chloride; and
g. water.

24. The method of claim 1, wherein the method further comprises administrating to the human subject a muscarinic receptor antagonist, a beta-adrenergic receptor agonist, an inhaled corticosteroid, or a combination thereof.

25. The method of claim 24, wherein the muscarinic receptor agonist is a long-acting muscarinic receptor antagonist (LAMA).

26. The method of claim 25, wherein the LAMA is aclidinium, darotropium, tiotropium, glycopyrrolate, or umeclidinium.

27. The method of claim 24, wherein the beta-adrenergic receptor agonist is a long-acting beta-adrenergic receptor agonist (LABA).

28. The method of claim 27, wherein the LABA is salmeterol, formoterol, indacaterol, vilanterol, olodaterol, abediterol or carmoterol.

29. The method of claim 24, wherein the inhaled corticosteroid is beclomethosone, budesonide, fluticasone propionate, ciclesonide, mometasone, or fluticasone furoate.

30. The method of claim 24, wherein the beta-adrenergic receptor agonist is salbutamol.
